(19) United States
(12) Patent Application Publication
(10) Pub. No.: US 2025/0242159 A1
(43) Pub. Date: Jul. 31, 2025

(54) SYSTEM AND METHOD FOR COMBINED PHOTOBIOMODULATION AND ELECTRICAL STIMULATION FOR EPILEPSY

(71) Applicant: Boston Scientific Neuromodulation Corporation, Valencia, CA (US)

(72) Inventors: Jianwen Gu, Richmond, TX (US); Changfang Zhu, Valencia, CA (US)

(73) Assignee: Boston Scientific Neuromodulation Corporation, Valencia, CA (US)

(21) Appl. No.: 19/040,399

(22) Filed: Jan. 29, 2025

Related U.S. Application Data
(60) Provisional application No. 63/626,737, filed on Jan. 30, 2024.

(51) Int. Cl. A61N 1/36; A61N 1/05; A61N 5/06

(52) U.S. Cl. CPC A61N 1/36064; A61N 1/36139; A61N 5/0601; A61N 5/0622; A61N 1/0531; A61N 1/0534

(57) ABSTRACT

A multi-modal method of stimulation to provide therapy for an epilepsy patient. A neuroprotective therapy including both an electrical modulation signal and an optical modulation signal is generated on a periodic or scheduled basis. The patient is monitored, and if a trigger condition arises indicating one of potential seizure onset, seizure onset, or an ongoing seizure, a symptom modulation therapy is initiated. The neuroprotective therapy and the symptom modulation therapy may have different targets and other parameters.

### CROSS-REFERENCE TO RELATED APPLICATIONS

This application claims the benefit of U.S. Provisional Patent Application Ser. No. 63/626,737, filed Jan. 30, 2024, which is incorporated herein by reference.

### BACKGROUND

Current treatments for epilepsy using implantable medical devices, including those adapted for application of electrical and/or magnetic therapy, focus on reducing seizures, and do not address underlying pathology. For example, it is known generally to detect signals indicating seizure onset or occurrence, such as by a mechanical sensor, and initiate a responsive or magnetic therapy to interrupt seizure-related signals. It is desirable in addition or instead to reduce seizure occurrence by addressing underlying pathology. It may also be desirable to provide seizure mitigation therapy in addition to pathology interruption treatments.

New and/or alternative treatments and systems for providing treatments incorporating photo-bio-modulation (PBM) and/or electrical stimulation are desired.

### OVERVIEW

The present inventors have recognized, among other things, that a problem to be solved is the need for new and/or alternative methods and systems for providing a neuroprotective effect addressing epilepsy. Further desirable is a system and/or method to provide seizure mitigation along with the neuroprotective therapy. The present invention relates generally to a multi-modal method of addressing epilepsy.

A first illustrative and non-limiting example takes the form of a system for treating a patient having a seizure condition comprising: a cortical lead adapted for positioning inside the skull of a patient and on the outside of the brain, the cortical lead including each of an electrode and an optical output device; a deep brain stimulation lead adapted for positioning inside the brain of a patient near the hippocampus or near a seizure focal location; a pulse generator coupled to each of the cortical lead and the deep brain stimulation lead, the pulse generator comprising a control circuitry, a sensing means, and a pulse generator circuit, the control circuitry configured to: a) use the pulse generator circuit and the cortical lead to issue each of first optical outputs and first electrical outputs to modulate a first neural target of the patient to provide neural protection and reduce progression of epilepsy; b) use the sensing means for identifying a triggering event indicating an elevated likelihood of actual or impending epilepsy seizure; and c) in response to the identifying, use the pulse generator circuit and the deep brain stimulation lead to issue second electrical outputs to modulate a second neural target of the patient to mitigate effects of the epilepsy seizure.

Additionally or alternatively, the control circuitry is also configured, in step c), to use the pulse generator circuit and the deep brain stimulation lead to issue second optical outputs to modulate the second neural target of the patient. Additionally or alternatively, the first neural target includes a portion of the cerebral cortex. Additionally or alternatively, the second neural target is the hippocampus. Additionally or alternatively, the second neural target is an epilepsy focus location. Additionally or alternatively, the first target is associated with generation of malignant electrical signals that can lead to epilepsy seizure, and the second target is associated with transmission of the malignant electrical signals.

Additionally or alternatively, the first electrical outputs are generated at a lower power density than the second electrical outputs. Additionally or alternatively, the first electrical outputs have at least one of a lower amplitude, a lower duty cycle, a lower pulse width, or a lower frequency than the second electrical outputs.

Additionally or alternatively, the first electrical outputs are issued using a first plurality of electrodes each spaced at least 2 cm apart, and the second electrical outputs are issued using a second plurality of electrodes spaced less than 1 cm apart.

Additionally or alternatively, the sensing means is configured to perform step b) by sensing electrical signals from the brain of the patient, analyzing the sensed electrical signals, defining an inter-ictal time period, and finding a divergence of the sensed electrical signals relative to a signal captured during the inter-ictal time period. Additionally or alternatively, the sensing means is configured to use at least one electrode on the cortical lead for sensing electrical signals from the brain.

Additionally or alternatively, step c) is performed by: the control circuitry initiating electrical modulation of the second neural target; the control circuitry using the sensing means to analyze the trigger condition and continuing the electrical modulation of the second neural target until the trigger condition ceases. Additionally or alternatively, step c) is performed by: initiating electrical modulation of the second neural target; continuing the electrical modulation of the second neural target while using the sensing means to analyze brain signals, until a stop-therapy condition arises.

Additionally or alternatively, the system includes a patient remote control adapted to communicate with the pulse generator, wherein the stop-therapy condition arises in response to: the patient using the patient remote control to indicate a seizure is not occurring; the patient remote control communicating to the pulse generator to request to stop therapy; and the pulse generator receiving the request to stop therapy. Additionally or alternatively, the stop-therapy condition includes a timeout. Additionally or alternatively, the stop therapy condition is determined by the control circuitry using the sensing means to determine that the patient no longer shows an elevated likelihood of actual or impending epilepsy seizure.

Another illustrative and non-limiting example takes the form of a method of treating a patient comprising: a) optically and electrically modulating a first neural target of the patient to provide neural protection to reduce progression of epilepsy; b) identifying a triggering event indicating an elevated likelihood of actual or impending epilepsy seizure; and c) in response to the identifying, electrically modulating a second neural target of the patient to mitigate effects of the epilepsy seizure.

Additionally or alternatively, step c) also includes optically modulating the second neural target of the patient to mitigate effects of the epilepsy seizure. Additionally or alternatively, step a) is performed using a cortical electrode positioned superficial to the brain; and step c) is performed using a deep brain stimulation electrode positioned inside the brain. Additionally or alternatively, step a) is performed on a periodic basis, and steps b) and c) are performed only when a triggering event is identified. Additionally or alternatively, electrical modulating in step a) is performed using two electrodes that are more than at least 2 cm apart, and step c) is performed using two electrodes that are less than one cm apart. Additionally or alternatively, step a) is performed at a lower power density than step c). Additionally or alternatively, step a) is performed with a lower output amplitude, duty cycle, pulse width, or frequency than step c). Additionally or alternatively, step b) is performed by sensing electrical signals from the brain of the patient, analyzing the sensed electrical signals, and finding a divergence from a brain signal captured during an inter-ictal time period.

Additionally or alternatively, step c) is performed by: initiating electrical modulation of the second neural target; analyzing the trigger condition and continuing the electrical modulation of the second neural target until the trigger condition ceases.

Additionally or alternatively, step c) is performed by: initiating electrical modulation of the second neural target; continuing the electrical modulation of the second neural target until a stop-therapy condition arises. Additionally or alternatively, the stop-therapy condition includes a patient indication that a seizure is not occurring. Additionally or alternatively, the stop-therapy condition includes a timeout. Additionally or alternatively, the stop-therapy condition includes a determination that the patient no longer shows an elevated likelihood of actual or impending epilepsy seizure.

Additionally or alternatively, step c) includes interrupting step a) and preventing step a) until step c) is completed.

Additionally or alternatively, the first target is associated with generation of malignant electrical signals that can lead to epilepsy seizure, and the second target is associated with transmission of the malignant electrical signals.

Additionally or alternatively, the first target is a seizure focus, and the second target is a part of the hippocampus. Additionally or alternatively, the first target is on or in the cerebral cortex, and the second target is a part of the hippocampus.

Another illustrative and non-limiting example takes the form of a system for treating a patient having a seizure condition comprising: a cortical lead adapted for positioning inside the skull of a patient and on the outside of the brain, the cortical lead including each of an electrode and an optical output device; a deep brain stimulation lead adapted for positioning inside the brain of a patient near the hippocampus or near a seizure focal location; a pulse generator coupled to each of the cortical lead and the deep brain stimulation lead, the pulse generator comprising a control circuitry, a sensing circuitry, and a pulse generator circuit, the control circuitry configured to: a) use the pulse generator circuit and the cortical lead to issue each of first optical outputs and first electrical outputs to modulate a first neural target of the patient to provide neural protection and reduce progression of epilepsy; b) use the sensing circuitry for identifying a triggering event indicating an elevated likelihood of actual or impending epilepsy seizure; and c) in response to the identifying, use the pulse generator circuit and the deep brain stimulation lead to issue second electrical outputs to modulate a second neural target of the patient to mitigate effects of the epilepsy seizure.

Additionally or alternatively, the control circuitry is also configured, in step c), to use the pulse generator circuit and the deep brain stimulation lead to issue second optical outputs to modulate the second neural target of the patient. Additionally or alternatively, the first neural target includes a portion of the cerebral cortex. Additionally or alternatively, the second neural target is the hippocampus. Additionally or alternatively, the second neural target is an epilepsy focus location. Additionally or alternatively, the first target is associated with generation of malignant electrical signals that can lead to epilepsy seizure, and the second target is associated with transmission of the malignant electrical signals.

Additionally or alternatively, the first electrical outputs are generated at a lower power density than the second electrical outputs. Additionally or alternatively, the first electrical outputs have at least one of a lower amplitude, a lower duty cycle, a lower pulse width, or a lower frequency than the second electrical outputs. Additionally or alternatively, the first electrical outputs are issued using a first plurality of electrodes each spaced at least 2 cm apart, and the second electrical outputs are issued using a second plurality of electrodes spaced less than 1 cm apart.

Additionally or alternatively, the sensing circuitry is configured to perform step b) by sensing electrical signals from the brain of the patient, analyzing the sensed electrical signals, defining an inter-ictal time period, and finding a divergence of the sensed electrical signals relative to a signal captured during the inter-ictal time period.

Additionally or alternatively, the sensing circuitry is configured to use at least one electrode on the cortical lead for sensing electrical signals from the brain.

Additionally or alternatively, step c) is performed by: initiating electrical modulation of the second neural target; using the sensing circuitry to analyze the trigger condition and continuing the electrical modulation of the second neural target until the trigger condition ceases.

Additionally or alternatively, step c) is performed by: initiating electrical modulation of the second neural target; continuing the electrical modulation of the second neural target while using the sensing circuitry to analyze brain signals, until a stop-therapy condition arises.

Additionally or alternatively, the system also includes a patient remote control adapted to communicate with the pulse generator, wherein the stop-therapy condition arises if: the patient uses the patient remote control to indicate a seizure is not occurring; the patient remote control communicates to the pulse generator to stop therapy; and the pulse generator receiving the request to stop therapy. Additionally or alternatively, the stop-therapy condition includes a timeout. Additionally or alternatively, the stop therapy condition includes the control circuitry using the sensing circuitry to determine that the patient no longer shows an elevated likelihood of actual or impending epilepsy seizure.

This overview is intended to introduce the subject matter of the present patent application. It is not intended to provide an exclusive or exhaustive explanation. The detailed description below provides further details.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 shows an illustrative DBS system implanted in a patient;

FIG. 2 illustrates details of a directional DBS lead;

FIG. 3 illustrates the details of an optrode lead;

FIG. 4 shows the intracranial placement an optrode lead;

FIG. 5 illustrates placement of two or more therapy devices intracranially;

FIG. 6 shows a device configured with optical output devices;

FIG. 7 shows an illustrative paddle lead with optical outputs;

FIG. 8 shows an illustrative implantable system with two separate therapy targets and therapy delivery subsystems;

FIG. 9 shows a block diagram for illustrative methods of treatment;

FIG. 10 illustrates selected brain anatomy; and

FIG. 11 shows an illustrative implantable pulse generator.

### DETAILED DESCRIPTION

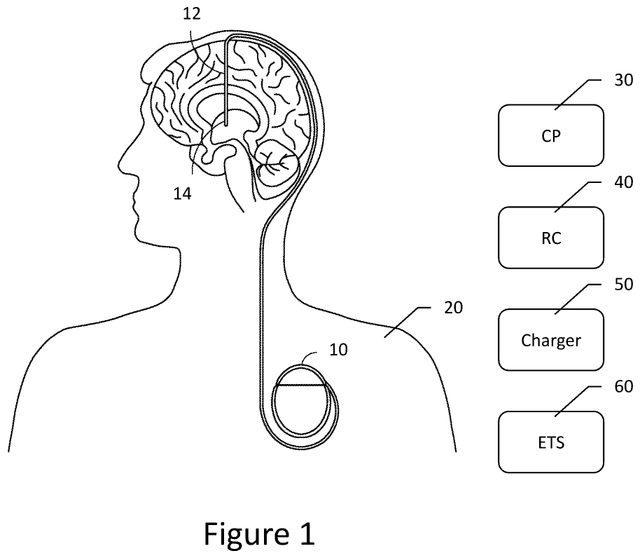

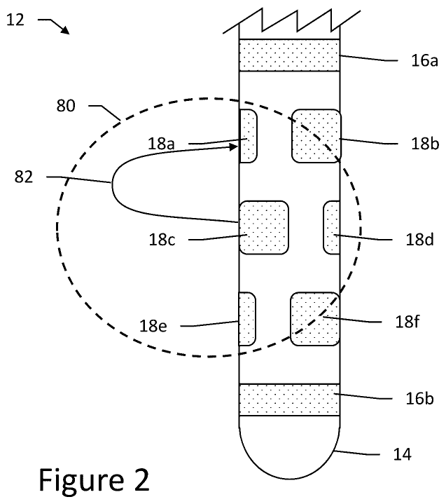

FIG. 1 shows an illustrative DBS system implanted in a patient. The system comprises a pulse generator 10, shown implanted in the pectoral region of a patient 20. The pulse generator 10 is coupled to a lead 12 which extends subcutaneously to the head of the patient 20, through a burr hole formed in the patient's skull, and then into the brain. In the example shown, the lead 12 includes a plurality of electrodes positioned near the distal end 14 of the lead, such as shown below in FIG. 2. The lead 12 may be placed at any suitable location of the brain where a target for therapy is identified. For example, a lead 12 may be positioned so that the distal end 14 is near the mid-brain and/or various structures therein that are known in the art for use in providing stimulation to treat various diseases.

DBS may be targeted, for example, and without limitation, at neuronal tissue in the thalamus, the hippocampus, the globus pallidus, the subthalamic nucleus, the pedunculopontine nucleus, substantia nigra pars reticulate, the cortex, the globus pallidus externus, the medial forebrain bundle, the periaquaductal gray, the periventricular gray, the habenula, the subgenual cingulate, the ventral intermediate nucleus, the anterior nucleus, other nuclei of the thalamus, the zona incerta, the ventral capsule, the ventral striatum, the nucleus accumbens, and/or white matter tracts connecting these and other structures. Data related to DBS may include the identification of neural tissue regions determined analytically to relate to side effects or benefits observed in practice. “Targets” as used herein are brain structures associated with therapeutic benefits, in contrast to avoidance regions or “Avoid” regions which are brain structures associated with side effects.

Conditions to be treated may include dementia, Alzheimer's disease, Parkinson's disease, seizure disorders including epilepsy, dyskinesias, tremors, depression, anxiety or other mood disorders, sleep related conditions, etc. Therapeutic benefits may include, for example, and without limitation, improved cognition, alertness, and/or memory, enhanced mood or sleep, elimination, avoidance or reduction of pain or tremor, reduction in motor impairments, and/or preservation of existing function and/or cellular structures, such as preventing loss of tissue and/or cell death. Therapeutic benefits may be monitored using, for example, patient surveys, performance tests, and/or physical monitoring such as determining seizure frequency, or monitoring gait or tremor, etc. Side effects can include a wide range of issues such as, for example, and without limitation, reduced cognition, alertness, and/or memory, degraded sleep, depression, anxiety, unexplained weight gain/loss, tinnitus, pain, tremor, etc. These are just examples, and the discussion of ailments, benefits and side effects is merely illustrative and not exhaustive.

The illustrative system of claim 1 includes various external devices. A clinician programmer (CP) 30 may be used to determine/select therapy programs, including steering (further explained below) as well as stimulation parameters. Stimulation parameters may include amplitude of stimulation pulses, frequency or repetition rate of stimulation pulses, pulse width of stimulation pulses, and more complex parameters such as burst definition, as are known in the art. Biphasic square waves are commonly used, though nothing in the present invention is limited to biphasic square waves, and ramped, triangular, sinusoidal, monophasic and other stimulation types may be used as desired. The CP 30 can be used by a physician, or at the direction of a physician, to obtain data from and provide instructions the pulse generator 10 via suitable communications protocols such as Bluetooth or MedRadio or other wireless communications standards, and/or via other modalities such as inductive telemetry.

A patient remote control (RC) 40 can be used by the patient to perform various actions relative to the pulse generator 10. These may be physician defined options, and may include, for example, turning therapy on and/or off, entering requested information (such as answering questions about activities, therapy benefits and side effects), and making (limited) adjustments to therapy such as selecting from available therapy programs and adjusting, for example, amplitude settings. The RC 40 can communicate via similar telemetry as the CP 30 to control and/or obtain data from the pulse generator 10. The patient RC 40 may also be programmable on its own, or may communicate or be linked with the CP 30.

A charger 50 may be provided to the patient to allow the patient to recharge the pulse generator 10, if the pulse generator 10 is rechargeable. Some pulse generators 10 are not rechargeable, and so the charger 50 may be omitted. The charger 50 can operate, for example, by generating a varying magnetic field to activate an inductor associated with the pulse generator 10 to provide power to recharge the pulse generator battery, using known methods and circuitry.

Some systems may include an ETS 60. The ETS 60 can be used to test therapy programs after the lead 12 has been implanted in the patient to determine whether therapy will or can work for the patient 16. For example, an initial implantation of the lead 12 can take place using, for example, a stereotactic guidance system, with the IPG 10 temporarily left out. After a period of healing, the patient may return to the clinic for therapy configuration and testing. The lead 12 may have a proximal end thereof connected to an intermediate connector (sometimes called an operating room cable) that couples to the ETS 60, and the ETS 60 can be programmed using the CP 30 with various therapy programs and stimulation parameters. Once therapy suitability for the patient is established to the satisfaction of the patient 16 and/or physician, the permanent IPG 10 is implanted and the lead 12 is connected thereto, with the ETS 30 then removed from use. Additional components, such as a remote monitor or bedside monitor may also be included.

The pulse generator 10 may include operational circuitry for generating output stimulation programs and/or pulses in accordance with stored instructions. Some examples of prior versions of such circuitry, as well as planned future examples, may be found in U.S. Pat. No. 10,716,932, the disclosure of which is incorporated herein by reference. Pulse generator circuitry may include that of the various commercially known implantable pulse generators for spinal cord stimulation, Vagus nerve stimulation, and/or DBS. Additional background and/or examples of the pulse generator 10, CP 30, RC 40, Charger 50, and ETS 60 can be found, for example and without limitation, in U.S. Pat. Nos. 6,895,280, 6,181,969, 6,516,227, 6,609,029, 6,609,032, 6,741,892, 7,949,395, 7,244,150, 7,672,734, 7,761,165, 7,974,706, 8,175,710, 8,224,450, and 8,364,278, the disclosures of which are incorporated herein by reference in their entireties.

FIG. 2 illustrates details of a directional DBS lead. The distal end 14 is shown, and a plurality of electrodes are shown as well. Two ring electrodes 16a, 16b (collectively ring electrodes 16) can be provided as shown, and a number of segmented electrodes are shown at 18a, 18b, 18c, 18d, 18e, 18f (collectively, segmented electrodes 18). Each electrode 16, 18 may be separately addressable in the system, such as by using a pulse generator having multiple independent current control (MICC) or multiple voltage sources.

MICC is a stimulus control system that provides a plurality of independently generated output currents that may each have an independent quantity of current. The use of MICC can allow spatially selective fields to be generated during therapy outputs. The term “fractionalization” may refer to how the total current issued by the pulse generator via the electrodes is divided up amongst the electrodes 16, 18 on the lead. It should be noted that the pulse generator canister may serve as an indifferent electrode or as a return electrode for therapy outputs. Alternatively, one of the lead electrodes (such as a ring electrode 16 or one or more of the segmented electrodes 18) may instead be used as a return electrode. Thus, for example, the electrodes 16, 18 on the lead may serve as cathodes while pulse generator canister serves as an anode during one phase of stimulation pulse delivery. In another example, some of the lead electrodes 16, 18 serve as cathodes, while other lead electrodes 16, 18 serve as anodes during one phase of stimulation pulse delivery.

Examples of electrical leads with segmented or directional lead structures are shown, for example and without limitation, in US PG Pat. Pubs. 20100268298, 20110005069, 20110078900, 20110130803, 20110130816, 20110130817, 20110130818, 20110238129, 20110313500, 20120016378, 20120046710, 20120071949, 20120165911, 20120197375, 20120203316, 20120203320, 20120203321, 20130197602, 20130261684, 20130325091, 20130317587, 20140039587, 20140353001, 20140358207, 20140358209, 20140358210, 20150018915, 20150021817, 20150045864, 20150021817, 20150066120, 20130197424, and 20150151113, and U.S. Pat. Nos. 8,483,237 and 8,321,025, the disclosures of which are incorporated herein by reference.

A directional lead as shown in FIG. 2 may be used to generate a stimulation field as illustrated at 80 in FIG. 2. The outer boundary of field 80 may be understood as representing an equipotential or equal field boundary, within which the electrical field is higher than an activation threshold, and outside of which the electrical field is below the threshold, for purposes of illustration. A population-based activation threshold may represent or approximate a voltage/field strength at which neural cells will activate; activation thresholds may be determined on a population basis, such as by relating to a voltage/field at which a 50% likelihood of activation of 50% of the cell population is determined; other boundaries/thresholds can be used. The shape of the field can be adjusted, as described variously in the references incorporated by reference above, by modifying the fractionalization of current issued via the electrodes. An electrical field as shown at 80 may be (roughly) generated by using electrode 18c as a cathode, and surrounding electrodes 18a, 18e, and 18d as anodes, for example. The actual characteristics of fractionalization may be more sophisticated than this simple example.

A related concept to the field shown at 80 in FIG. 2 is that of stimulation field modeling (SFM). In SFM, the tissue is modeled, for example, using finite element models in which the lead body is treated as an insulator, surrounded by a thin encapsulation sheath, and further surrounded by neural tissue. The neural tissue may be modeled as isotropic and homogenous, though more sophisticated modelling can also be used if desired. A set of model volumes are defined around the lead, breaking up the space into small blocks, each of which can be analyzed within the model. The outer boundaries of the SFM can be determined using a population-based activation threshold. The result can be that at a given stimulation current, an SFM can be generated as a three-dimensional surface surrounding a portion of the lead and encompassing a volume of neural tissue. Field 80 may be, for example, understood as a two-dimensional representation of a slice of the SFM.

The positioning of the lead (and related SFM) relative to target and/or non-target (side-effect) neural structures may be determined for a given system using imaging modalities such as X-ray, CT scan, PET scan, MRI, f-MRI, etc., to identify lead position in the patient. Imaging system data for the patient may be used to estimate locations of neural structures in the patient, relative to the identified lead position. The imaging data may be merged or overlapped with general anatomical knowledge, such as from a brain atlas, which can help identify the particular structures that can be identified from imaging data. The use of SFM in association with a directional lead can allow therapy planning to precisely stimulate targeted tissue structures while avoiding excess stimulation of non-target tissue and limiting stimulation of structures associated with adverse side effects.

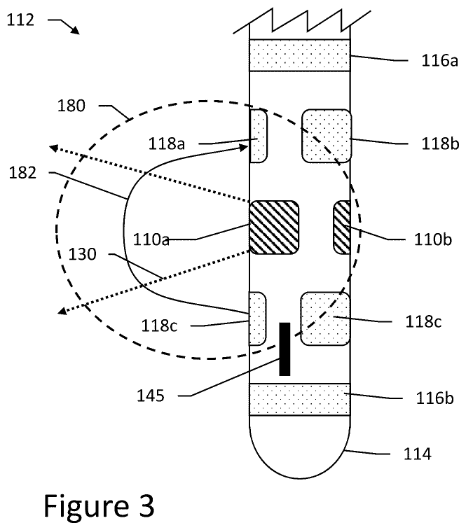

FIG. 3 illustrates details of an optrode lead 112 which comprises of a plurality of electrodes 116a, 116b (collectively 116), 118a, 118b, 118c, 118d (collectively, 118) and also includes optical devices 110a, 110b (collectively optical devices 110). The electrodes are used to issue current/voltage to electrically stimulate 180 the target neural structure. As used herein, an “optrode lead” 112 includes each of optical devices 110 and electrodes 118. The optical devices 110 produce a directional optical output 130 which in turn causes photo-bio-modulation (PBM) of the target. PBM refers to induced changes to cellular activity, traits, or longevity as a result of irradiation with selected wavelength(s) of light. A radiopaque marker 145, which can be detected via X-ray to indicate position and direction of the electrodes 116, 118 and optical devices 110 relative to tissue, may be provided as well on the optrode lead 112.

PBM has been applied to promote healing and regeneration of various tissues and has been used in multiple fields including sports medicine, orthopedics, dentistry, diabetes, and dermatology. Therapeutic benefits may include, for example, and without limitation, improved tissue repair and regeneration, reduced inflammation, improved management of acute and chronic pain, enhancing mitochondrial function, reducing oxidative stress of the target, and enhancement of circulation and vasodilation. Such benefits can be produced with minimal side effects and damage to the target.

As it pertains to the neuroprotective effects of PBM, an increase in vasodilation may improve the circulation of blood to the target. Such enhancement promotes tissue healing and repair, improve oxygen and nutrient delivery to the target. Additionally, PBM may enhance mitochondrial activity and increase ATP production, directly affecting neural cells themselves in addition to providing the secondary effect of increased circulation. By promoting ATP production, PBM can help support the energy needs of and neurotransmission by targeted neurons and maintain cellular metabolism and homeostasis of ion pumps. By promoting ATP production, PBM can contribute to the health, survival, and functionality of the target neural tissue.

The effectiveness of PBM therapy to the particular target may be influenced by the choice of wavelength of light 130 emitted by the optical devices 110. Optical devices 110 may use a light source to generate a full field optical output around the lead, or to generate a spatially selective directional optical output 130 giving rise to PBM of the relevant target. The light source may be placed locally at the distal end of the lead or distantly at the proximal end of the lead. The light source may be, for example, a laser diode or a light-emitting diode (LED). Light delivery systems, such as optical fibers, can be employed to deliver the light (whether collimated or not) in such a way to direct the optical output 130 to the target with precision. Some examples may use an array of vertical cavity surface emitting lasers, for example. Some examples, rather than collimating light for a highly directional output, may use a convex surface to disperse light, if desired.

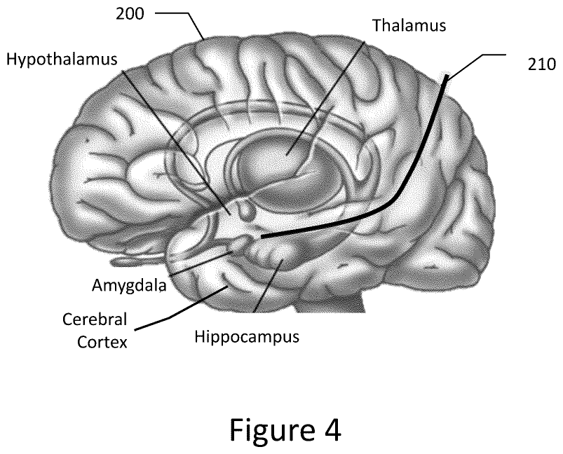

As shown further in the examples of FIGS. 3-4, the light source is placed near or in contact with the targeted tissue 210, thus allowing the optical output 130 to penetrate the target and induce the physiological reactions that generate the therapeutic benefits of PBM. Red light or near-infrared light, and/or other wavelengths of light, for example, in the wavelength range of about 600 to about-1300 nm may be used for inducing the desired therapeutic benefits of PBM. Other stimulation parameters that may influence the effect of PBM include, but is not limited to, the energy intensity of the light source, the power intensity and/or power density of the optical device 110, whether a continuous wave or pulsed light is issued, and the duration of the therapy. A plurality of selective wavelength sources may be used simultaneously, sequentially, singularly, or in other combinations or patterns, as desired.

The plurality of electrodes 116, 118 and optical devices 110 in FIG. 3 are arranged around the circumference of the lead 112, allowing the lead 112 to stimulate any target near the lead 112 without having to re-orient the lead 112. In use, the active optical devices 110 and electrodes 118 may be spatially aligned along the same longitudinal position of the lead 100, or, in the alternative, may be offset from one another. When aligned along lead 100, the electrodes 118 and the optical devices 110 are positioned in such a way to stimulate the same target. A marker 145 may be placed on the lead 112 to indicate the position and alignment of the lead 112 needed for precise and accurate optical and electrical stimulation 130, 182 of the target.

The optical devices 110 may be separately addressable so as to emit specific spatially selective directional optical outputs 130 suited to stimulate the chosen target. For example, optical devices 110 may emit a pre-determined, uniform optical output 130 or may be used with a compatible PBM control system to separately address the optical devices 110. PBM control systems are configured to adjust the stimulation parameters of PBM therapy best suited to bring about the desired therapeutic benefits to the target. Stimulation parameters that may be controlled by the PBM control system include, but are not limited to, selection of the wavelength of the optical output 130, the intensity and dosage of the optical output, the pulsing setting (PW, rate, and duty cycle etc) of the output, the duration and timing of optical outputs, and the schedule of PBM therapy. Control systems may include computer-controlled systems or programmable optical devices 110 to control the optical output 130.

In some examples, each light source 110 on the lead and/or at the end of the lead 112 may be a transducer, such as a laser or diode or other light emitting device, each (separately) coupled electrically to the pulse generator by electrical connections through the body of lead 112. A common ground wire may be used by plural light transducers, as desired. Alternatively, one or more light sources may be provided in a pulse generator (not shown in FIG. 3), and coupled by a light guide, such as an optical fiber, through the lead.

Another design may be understood by reference to FIG. 3, with somewhat different understanding of certain parts. The tip 114 may be an optical element in addition to or in place of the light sources 110. For example, the tip 114 may include a dome shaped optically transparent material, acting as a dispersive lens that can provide an optical output. The dome shape and/or lens-like capability is optional; any suitable design to allow an optical output to be delivered at the tip can be used, if desired.

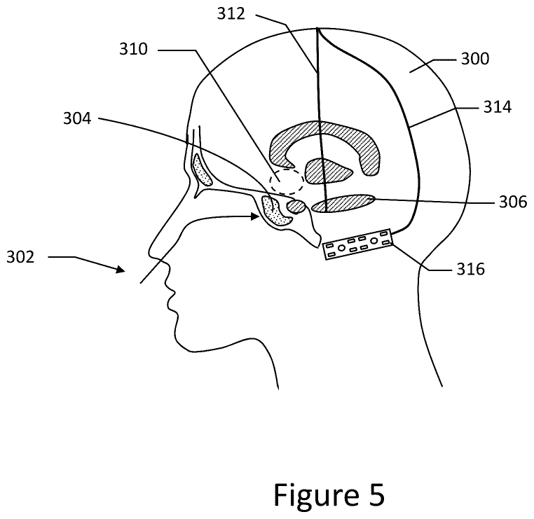

Referring briefly to FIG. 5, another optrode design is shown at 316 in the form of a paddle-type design. The two round circles represent first and second optical output devices, which may each be identical in design to one another, or may each be different. For example, one optical device may be configured for a first wavelength, and the other optical device may be configured for a second wavelength. In another example, one optical device may output light, and the other may detect light allowing tissue measurements to be obtained if desired, or allowing monitoring of optical output therapies. Eight small rectangles represent electrodes on the optrode 316. Alternatively, element 316 can carry just an electrode array, or just an array of optical output and/or sensing devices. Thus, a paddle design may be an alternative to the design shown in FIG. 3. In some examples a paddle design may be placed on the brain, such as over the cerebral cortex, or between tissue layers, tissue types, or into tissue folds, as desired. A directional lead design as in FIG. 3 may instead be used for positions inside of a given structure, such as locations adjacent, along or in the hippocampus, the NBM and/or basal forebrain.

An optrode lead positioned as illustrated at 316 may provide cortical stimulation, but may be positioned, for example, in a lateral position reached, if desired, by advancing a paddle lead between issue layers to achieve a subdural or epidural position, as desired. Light stimulation can then be generated and directed toward the brain tissue, from either epidural or subdural positions. Such positioning may be advantageous relative to other approaches, such as the transcranial approach, insofar as the cranium block much of the light stimulus that can be applied. Positioning may require the use of a curved forceps or other introducer tool to position the paddle as desired from a single burr hole through which lead 312 may also be placed. Alternatively, a second burr hole may be used for placement of the optrode 316 in addition to that used for lead 312. The paddle-like design can be kept relatively thin (no more than a few millimeters thickness), with a width that will allow secure placement without cutting tissue during any advancement of the lead 316, such as in the range of 5 to 20 mm width. Wider, narrower, or thicker designs may also be used.

FIG. 4 illustrates the placement of a lead or optrode lead 210 at a target location inside of brain 200. In particular, here, the lead 210 is shown alongside the hippocampus, along the upper or dorsal margin thereof. The optrode lead 210 may instead be beneath or ventral to the hippocampus, for example between the hippocampus and the cerebral cortex, or may be positioned along the left or right side thereof. In another example, two optrode leads are positioned, one to the left and the other to the right, of the hippocampus. These positions may be of interest for epilepsy treatments, as epilepsy has been associated with neuronal death in and around the site of abnormal neuronal firing, typically around the hippocampus. The abnormal neural firing in the region of the hippocampus may also be associated with or linked to loss of inhibitory interneurons in the same region. Other targets may include the subthalamic nucleus, the anterior and centro-median nuclei of the thalamus, and the cerebellum.

The selection of the region or subregion target is dependent on a variety of factors, including, but limited to, the desired therapeutic effect and the neural structure of the patient. In some examples, if seizure foci are identified, therapy may be directed to seizure foci as well or instead of these other anatomically targeted locations. Therapy may be directed to and across a region of tissue as well. For example, an optrode may be positioned dorsal and adjacent the hippocampus and used to apply optical therapy to arrest neurodegeneration in the hippocampus associated with progression of epilepsy, with an electrode or electrode array outside of the ventral portion of the cerebral cortex nearest the hippocampus, so that electrical therapy can also be directed through both the hippocampus and the adjacent portion of the cerebral cortex. In another example, the optical therapy may be generated from outside the cerebral cortex at a ventral position, with electrical therapy generated along the hippocampus from a lead adjacent to the hippocampus, dorsal or ventral thereof as desired, with the electrical therapy issued on an episodic basis to modulate of malignant or abnormal signaling and the optical therapy generated on a periodic or continuous basis to arrest disease progression and/or encourage cell vitality.

In some examples, a tissue region/volume that is stimulated by the electrical stimulation 180 may overlap at least a portion of a tissue region/volume that is also stimulated by the optical output, to increase therapeutic effect or to overlap two different mechanisms of therapy effects. Stimulation may be simultaneous both spatially and temporally, if desired. In other examples, electrical stimulation and optical stimulation may be interleaved, such as by temporally offsetting one relative to the other.

In some examples, optical stimulation of a region may be coordinated with electrical therapy designed to modulate signal transmission in neural tissue around the region of optical stimulation. This may reduce side effects of the stimulation from being transmitted to other parts of the body, if desired.

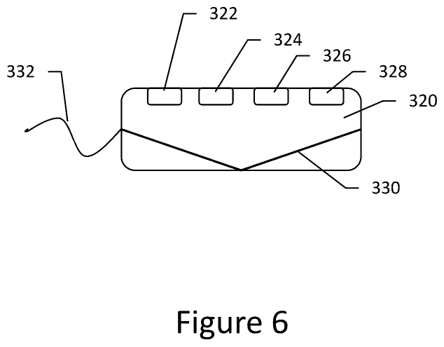

FIG. 5 illustrates placement of two or more therapy devices intracranially. For example, FIG. 5 shows a path for placement of a device in a sinus of a patient 300. Here, an intranasal path for placement of a device is illustrated at 302, extending to the sphenoid sinus 304. A standard DBS lead 312 maybe positioned as shown, with optical stimulation provided from a different location in a less invasive manner. For example, a device as shown in FIG. 6, below, may be positioned in a sinus cavity of a patient. The use of the sphenoid sinus 304 is illustrative, and may be beneficial for its proximity to the basal forebrain and NBM, allowing region 310 to be stimulated with optical energy. Some or all of the hippocampus 306 may also be subject to optical stimulation from the intranasal position 304. In some examples, the intranasal positioning may be used to for patients having a seizure focus identified in the frontal lobe.

In some examples, the system may include both the DBS lead 312 and a second lead 314 extending to an optrode device 316. The optrode device 316 may include one or more optical output devices (shown as circles) and a plurality of electrodes (small rectangles in the figure), and may be understood as having a position on the outside surface of the brain, beneath the skull, generally positioned on the cerebral cortex. The location of the optrode 316 may be lateral to or ventral to the hippocampus 306, as desired. Therapy from this location may be considered superficial, as opposed to deep brain stimulation from a lead or optrode located alongside the hippocampus. In some such examples with both a DBS and a superficial intracranial lead, the illustrative intranasal device may be included, while in others the intranasal device is omitted.

For epilepsy, the stimulation target may include the hippocampal formation or anterior nucleus of the thalamus, or other DBS targets. For example, epilepsy treatments may target the anterior and/or centromedian nuclei of the thalamus, the subthalamic nucleus, and/or the cerebellum. Other targets, including seizure foci, may be selected.

A device as shown in FIG. 6 may be placed. For example, the device 320 may include a plurality of optical output devices 322, 324, 326, 328, such as light emitting diodes, lasers, etc. As few as a single optical output device may be provided, or as many as are desired; four are shown for illustration. For optical stimulation related to epilepsy having identified seizure foci in the forebrain, it would be desirable to direct the optical devices toward the forebrain when emplaced.

Placed in contact with the nasal mucosa, optical stimulation may be administered through the nasal cavity via the device 320. The nasal mucosa has a high degree of vascularity and high amount of blood vessels. These features of the nasal mucosa allow the delivery of an optical output targeting neural tissue in the brain. The lead, as shown at 312 in FIG. 5, may be a standard DBS lead if desired. In an alternative, the lead 312, or lead 314, may include an optical sensor configured to determine whether output optical energy from a device 320 is reaching the target tissue, allowing calibration of power level, wavelength, and/or spatial selection of optical sources. For example, FIG. 3 may be understood as alternatively showing optical sensors at 110a, 110b, facilitating detection that would confirm the optical outputs from device 320 provide desired levels of power/energy, etc. The intracranial lead can also be used to issue electrical therapy signals.

Because intranasal delivery of the optical 130 stimulation to the forebrain is indirect in its nature, considerations in determining the stimulation parameters may not be consistent with the stimulation parameters applied to intracranial leads. Adjusting the stimulation parameters may be necessary to limit stimulation of avoid regions due to the limited precision and localization of intranasal stimulation and the differing anatomy and nasal conditions of the patient 20.

In FIG. 5, the device can be inserted through the nose or throat, and placed in or in proximity to the sphenoid sinus 304. The sphenoid sinus 304 may be selected because it is both accessible through intranasal placement and proximate to the forebrain. However, selection of the intranasal placement of the device 320 is not limited to the sphenoid sinus 304. Other paranasal sinuses (the frontal, axillary, and/or ethmoid sinuses) may be used, particularly if seizure foci are identified in brain tissues near any of these sinuses.

In still another example, the device 320 of FIG. 6 may include one or more markers 330, such as radiopaque markers that can allow a determination of the orientation of the device 320 once implanted. The device 320 may include a tether 332 that can be used for device retrieval; a hook or other structure to which a retrieval device can be attached may be included instead or in addition to the tether 332. Device 320 may be internally powered, such as by a battery, if desired. Alternatively, the device 320 may be externally powered, such as by including an inductive or other transducer to receive power from magnetic fields, RF fields, ultrasound signals, etc., from an external device.

In another example, the device 320 may be used in conjunction with an optrode as shown in FIG. 3 for purposes of configuring device parameters. For example, device 320 may have one or more optical receivers at any of 322, 324, 326, 328, and may be placed intracranially in a sinus, such as the sphenoid sinus 304. The optrode may then be activated and optical energy issued while the optical receiver on device 320 is used to determine whether light energy is being transmitted to the correct tissue at desired amplitudes. Following confirmation that therapy is reaching targeted tissue, the device 320 may be removed, if desired.

In another alternative, optical and/or electrical sensors may be positioned on a catheter, a guidewire, or other intraluminal device that can be threaded through the vasculature, for example, to a desired location with the aid of fluoroscopic guidance. With the optical and/or electrical sensors positioned as desired, the optical and/or electrical therapy can be generated, and the sensors used to confirm that therapy is reaching target tissue in desired quantities/amplitudes/power levels.

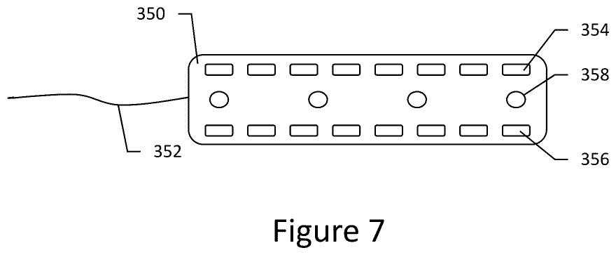

In some examples, a paddle lead may be used to provide electrical and/or optical outputs. An example is in FIG. 7. Here, the paddle lead includes a paddle body 350, typically formed of a biocompatible and largely dielectric polymer, with wire connections inside to each of a plurality of electrodes. The example shown has 16 electrodes arranged in two rows at 354 and 356. Further, there may be one or more optical output devices as shown in the line at 358. In the example, four optical output devices are provided. Each optical output device 358 may be independently addressable, as may be each of the electrodes 354 and 356. The illustrative version shown in FIG. 5 is generally similar in layout, but has fewer electrodes 354 and optical devices 358.

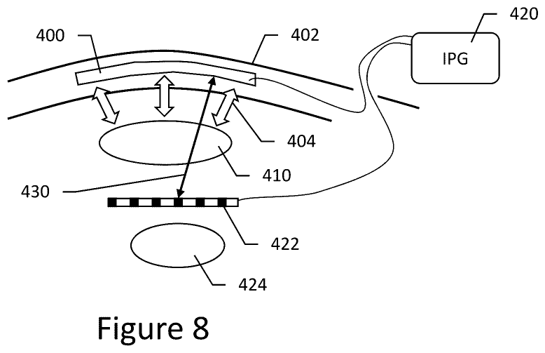

A paddle or a standard lead, or an optrode, may be used to provide a therapy as shown in FIG. 8. Here, a paddle lead is shown at 400, inside a bony structure 402, which may be the skull or a portion the spine, for example and without limitation. Therapy outputs are directed as shown at 404 toward a target structure at 410, which may be, for example, an occipital node, the spinal cord, a cortical structure, etc. Therapy outputs 404 may be optical, electrical, or both optical and electrical, as desired.

The system may include an implantable pulse generator as shown at 420, implanted, for example and without limitation, in the region of the neck, shoulder, or upper chest of the patient. In some examples, the system may also include a deep brain stimulation lead as indicated at 422, having a design, for example, as shown above in FIGS. 2 and/or 3, or using any other suitable design for deep brain stimulation leads. The positioning of either lead 400, 422 may be performed by first executing a preoperative study of the patient to identify one or more locations where seizures are best sensed and a seizure focus.

In some examples, known methods, including MRI, and/or electrical detection of signals occurring during seizures (or combinations thereof) may be used to estimate locations of one or more foci of a patient's seizures. Then the lead, whether the lead is an optrode having both electrical and optical outputs, or only an electrical lead or only an optical lead, may be positioned to target the location of the epilepsy seizure foci.

The seizure focus is the site in the brain from which the seizure originated. A seizure focus can be identified through imaging and EEG recording by identifying regions of functional deficit during the interictal period, regions that generate interictal spikes, regions responsible for the ictal symptoms, regions from which the seizure is triggered, and/or regions of structural damage. Any such region can be selected; it may be that a location for lead implantation can be selected to corresponding to a location of two or more of these markers of seizure focus. Many seizures also depend on nodal interactions that permit spontaneous network excitability and behavioral expression. To the extent nodal interactions and/or network regions can be identified that serve to transmit signals to which seizure symptoms can be attributed, lead placement may be chosen to interrupt such signal transmission, rather than or in addition to identifying seizure a focus location for lead implantation. The physician may weigh, for example, the ease or difficulty of accessing a particular location during lead implant, as well as the potential for side effects when therapy is issued to mitigate epileptic seizure. For example, if multiple possible implant positions are identified, surrounding neural structures may be analyzed to help rule out any potential lead locations adjacent to structures that may be associated with side effects.

In an illustrative example, the paddle lead 400 (or in the alternative, a strip lead, linear lead, etc.) is implanted in a cortical position and is used for sensing electrical signals in the brain, seeking to detect signs of an epileptic seizure, whether precurser signals of a potential seizure, or signals indicative of seizure onset, or signals indicating ongoing seizure. Triggers will typically be identified by the use of the electro-encephalogram (EEG), though other triggers may also be used. The EEG may, for example, be subject to frequency selective filtering and/or decomposition (principal components analysis, wavelet transform, etc.) to identify those signal components of highest interest for a particular patient. For example, an EEG captured during a non-seizure time period may be compared to the EEG captured just before, at onset of, and during a seizure, to identify any components that vary from one patient state to the next. One marker is the interictal epileptiform discharge, which may present as spikes, polyspikes, etc. High frequency oscillations, and connectivity analysis, have also been suggested for use in identifying seizure occurrence and/or onset. Identification of any such signal can be used as a trigger for seizure mitigation therapy. Higher frequency oscillations (in the range of about 80 to 500 Hz for example), may also be an EEG biomarker for triggering anti-seizure therapy.

In some examples, both seizure identification and mitigation can be performed in a single lead. Further, a second lead, located elsewhere in the brain, may be used to issue neuroprotective therapy at a different location. This combination may reflect the understanding that root cause of a seizure may lie in a first brain region, but the transmission (connectivity) that allows the root cause to create a seizure effect may lie in a second (or more) brain region. Neuroprotective therapy may be used to treat the brain region in from which the original signals begin to generate, and/or are amplified by connectivity. Mitigation therapy may be used to interrupt transmission of the signals that lead to epilepsy seizure symptoms. Thus, neuroprotective therapy is, in some examples, a therapy issued generally at lower intensity, when compared to mitigation therapy that provides higher power outputs. Both therapy types may be used in a relatively infrequent manner or low-duty cycle, though it will again be understood that the neuroprotective therapy can occur at a regular interval and is therefore periodic, while the mitigation therapy is episodic and triggered.

Some examples may provide therapy for seizure mitigation using, for example, a frequency of about 200 Hz, a pulse width of about 160 microseconds, delivered in bursts of 100 milliseconds, with pauses between bursts of, for example, 100 milliseconds to 10 seconds, until the signal that caused the trigger ceases to be detected. Other approaches may be used. Seizure mitigation therapy amplitude may be, for example, in the range of up to 12 mA per electrode, with titration at the option of a treating physician. Clinical study of such a mitigation signal found that, in selected patient populations having epilepsy, actual use was in the range of about 3-8 minutes per day, on average for most patients.

More generally, therapy parameters may vary. The frequency used may be in the range of about 1 Hz to 1500 Hz, or more or less, as desired; some systems may be limited to 2 Hz to 255 Hz, for example. Some examples directed to seizure mitigation may use a frequency in the range of 60 to 250 Hz. Pulse width can range, for example, from 10 to 1000 microseconds, or 20 to 450 microseconds, and may be selected in some examples for seizure mitigation in the range of 60 to 200 microseconds. Amplitude for a current controlled output may be in the range of about 0.1 mA up to 20 mA, for example and without limitation. A voltage controlled system may use therapy amplitudes of up to about 10 V, as desired. Cycling on and off of therapy can be used, as desired.

Neuroprotective electrical therapy may be within any of the preceding ranges. In some examples, a voltage-controlled system may use an amplitude in the range of 1-10 volts, cycling on/off at about 1 minute (1-3 minutes range, for example) on, and 3 minutes (2-5 minutes range, for example) off. Pulse width may be, for example, about 60 to 150 microseconds, with a frequency of about 70 to 200 Hz. Other parameters can be used.

The neuroprotective electrical therapy is then paired with an optical therapy output. Light may be emitted, for example and without limitation, at one or more wavelengths in the range of 500 to 2000 nanometers. Power level on the light output can range, for example, up to about 100 milliwatts on average, with peak power higher during pulsing outputs. These numerical examples are not intended to be limiting. A pulsed light output can be generated at one or more wavelengths using a vertical cavity surface emitting laser (VCSEL), or a light emitting diode, located on a paddle as shown in FIG. 8, and/or on a lead as shown in FIG. 3. Alternatively, the light output can be generated inside the IPG by a laser, light emitting diode, etc. and conveyed to the distal end of a lead by an optical fiber inside the lead. Use of an optical fiber in the lead may add to lead complexity or failure rates, and may also limit, for example, the flexibility or minimum turn radius of a lead when the lead passes from outside the skull to the interior thereof through a cranial port, thus some examples use an optical transducer (VCSEL, light emitting diode, etc.) at the distal end of the lead. If a non-directional, but wavelength specific output is desired, a dispersing lens may be used.

In some examples a combination of two targets may be addressed for different purposes. For example, a first (electrical only, or PBM) lead may be positioned near an epilepsy seizure focus location, and a second lead (optical only, or PBM) may be positioned at a second location, such as near a location in which neural function is to be protected, for example, such as near the hippocampus and/or near the thalamus. For such an example, long term therapy may be delivered on a continuous, but low duty cycle basis to provide a neuroprotective effect at the first location, and short-term therapy in response to a potential or actual seizure can be provided temporarily to prevent, terminate, or mitigate seizures at the second location. The long-term therapy can include an optical component, or may include both optical and electrical stimulation or modulation, while the short-term therapy may be only electrical, or may include both optical and electrical stimulation or modulation, as desired.

In one particular example, a system generally as shown in FIG. 8 may be used. A first lead 400 is implanted in a cortical position, beneath the skull (shown at bony structure 402), and is used to both sense signals from the brain and, at least periodically, to issue output stimulation including at least light outputs to the brain. The light outputs can be used to irradiate a relatively large volume of tissue in the brain from such a position. The light output is paired with a first electrical output, generated by either lead 400 or lead 422. Lead 422 may be considered a DBS lead, and can be located near the hippocampus. The paired light and first electrical output is used for neuroprotective purposes, and is generated in an example for about 1 to about 30 minutes each day, preferably during or just before waking of the patient. The volume of tissue subject to light therapy may overlap with, or may be different from, that receiving the first electrical therapy. Sensed signals of the EEG are also monitored. When a trigger is identified, indicating potential or actual seizure onset, or an ongoing seizure, a second therapy is triggered. The second therapy is an electrical therapy generated using the second lead 422. At least one of the frequency, pulse width, and amplitude of the second electrical therapy exceeds that of the first electrical therapy, such that the power output with the second electrical therapy is at least 25%, or, at least 50%, greater than the power output with the first electrical therapy. In some examples, one or both of the first therapy or second therapy may be directed as illustrated at 430, between the cortical lead 400 and the DBS lead 422, if desired.

In some examples, the first electrical therapy may be issued with a duty cycling during active use of less than about 40%, and more often, less than 30% (for example, one minute on, and two minutes off); within the first electrical therapy, any of a tonic or burst therapy may be used, with ramping as is know in the art. The second electrical therapy may be generated with a duty cycle of about 50% in some examples. Rated relative to the total duration of the first electrical therapy and the second electrical therapy, the average power of the second electrical therapy may be twice that of the first electrical therapy, in some examples.

Stimulation may target other locations in the body, including the cervical or thoracic spinal cord, and/or the trigeminal nerve, as desired. The lead, whether the lead is an optrode having both electrical and optical outputs, or only an electrical lead or only an optical lead, may be positioned to target the cervical or thoracic spinal cord, or the trigeminal nerve.

In some examples, electrical signal delivery may have a non-stimulating effect, such as to down-regulate one or more neural networks or activities in the tissue subject to the electrical pulses. Thus, some examples herein may refer to electrical modulation, which means the issuance of electrical signals that may include both electrical signals having stimulatory effects as well as down-regulating effects. The same is true for optical stimulation. Optical outputs may cause stimulation and/or down-regulation of neural activity. Optical modulation encompasses optical outputs that cause stimulation as well as optical outputs that cause down-regulation of neural activity.

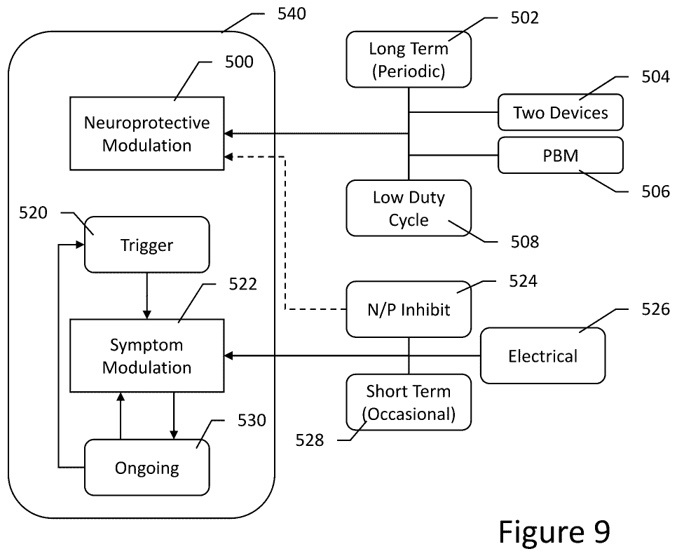

FIG. 9 is a block diagram that is useful to explain several examples. A neuroprotective modulation is generated in block 500, part of an overall therapy 540. This modulation may be a long term and/or periodic, as indicated at 502, such as by occurring on a scheduled basis (thrice, twice or once daily, alternating days, weekly, or at other interval/period). Two devices may be used, as indicated at 504. This may mean the use of two separate devices, such as a first device in a nasal sinus (FIG. 6) operating along with a second device located elsewhere in the patient, such as using a cortical, deep brain, or other lead, where each device is separately powered, where the two devices are both scheduled to operate in overlapping or identical time frames. In other examples, two devices 504 may mean two leads located at different locations (FIG. 8, for example) in the patient, such as a cortical lead and a deep brain lead located near another target, such as a seizure focus or the hippocampus, or another structure.

The output for neuroprotective therapy may include photobiomodulation (PBM) 506, and include both electrical and optical outputs, whether addressing the same or distinct volumes of tissue. PBM may include, for example, electrical signals along with optical signals, delivered for a period in the range of 1 second to 1 hour, more preferably about 2 minutes to about 30 minutes. The optical signals may have wavelengths in the range of about 500 to about 2000 nanometers, or about 600 to 1000 nanometers, or more or less, as desired, with power levels ranging from 10 microwatt to 1000 milliwatts, for example, delivered continuously or as tonic or burst-type pulses; multiple wavelengths can be issued if desired, simultaneously or in any pattern or sequence. The long-term neuroprotective modulation may have a low duty cycle 508, as will be further explained below.

The overall therapy 540 also includes a triggered symptom modulation therapy 522. The trigger 520 may include sensing of any indication of an epilepsy seizure onset. A trigger may include a patient generated communication, such as via a patient remote control or external device (which may be a smart phone), wherein the patient may tap an icon or otherwise indicate that the patient is experiencing a seizure. A trigger can be detection of tremor or movement associated with a seizure, which can be detected using a wearable device (movement trackers, for example), or by an accelerometer provided in an implantable pulse generator. In several examples, a trigger may be provided by detecting electrical signals in the patient's brain (EEG) and identifying seizure onset due to specific abnormalities in the EEG. These may include, for example and without limitation, interictal epileptiform discharge, which may present as spikes, polyspikes, etc., and/or high frequency oscillations, and/or connectivity analysis.

Once a triggering event is identified at 520, the method initiates symptom modulation at 522. Neuroprotective outputs may be inhibited as indicated at block 524, if desired, stopping the neuroprotective modulation from occurring at the same time as the symptom modulation. Block 524 is optional. The symptom modulation may be primarily electrical in nature, though optical outputs may also be generated, as desired, and as indicated at 526. The symptom modulation can be an occasional and short-term therapy, as indicated at 528. In some examples, symptom modulation occurs for a fixed period of time, in the range of about 1 minute to about one hour, or more or less. In other examples, as indicated at 530, the symptom modulation may be ongoing until the signal detected during the triggering step 520, or another signal indicative of ongoing seizure activity, ceases. In still other examples, the ongoing element at 530 may wait for a stop therapy marker. For example, the patient may indicate that no seizure is occurring (whether or not one actually occurred) using a patient remote control, and that may serve as the marker to stop therapy. Resumption of a “normal” pattern of EEG may be a stop-therapy marker, where “normal” may be physician defined or may be determined by comparing a received EEG signal, or a frequency analysis thereof, to a recorded EEG signal obtained during an interictal or postictal time period for a prior event. Some examples may use an ongoing loop between blocks 522 and 530 until either a timeout occurs, at for example, 5 to 30 minutes from onset, or until a stop-therapy marker is received or identified.

An optical detector may be provided to identify other indications of seizure onset, such as to determine oxygenation status of brain or other neural tissue. A drop or other change in oxygenation status of the neural tissue may be a trigger for a seizure mitigation signal, if desired.

The electrical signals issued at block 500 may be different, in effect, from those at 522. In some examples, the electrical signals at block 500 may be intended to provide stimulus or modulation across a larger volume of tissue, and so spaced apart electrodes may be used. Symptom modulation at 522, in some examples, targets a smaller volume, and therefore uses closer spaced electrodes, so that even if the signal amplitude is the same in each of blocks 500 and 522, the power density is higher for symptom modulating signals issued at 522. Other examples may operate differently. In some examples, the signals at 500 are issued without requiring a triggering event, and in some examples are paired with an optical output. A volume of tissue receiving optical outputs may be the same as or may overlap with the volume of tissue subjected to the electric outputs of block 500.

The long-term neuroprotective modulation may have a low duty cycle 508. For example, when therapy is actually called for the neuroprotective modulation at 500 may be for a period of less than one hour of every twenty-four hours. When a trigger occurs at 520, the symptom modulation 522 is enabled, and will use a much higher duty cycle, in the range of 10% to 99%, for example, and more preferably in the range of about 25%-75%. By duty cycle, here, the meaning is based on the period in which an active therapy output comprising pulses with quiescent periods is generated, relative to total rest periods between such active therapy outputs. Alternatively, in some examples, the long-term neuroprotective modulation may be issued more or less continuously with a one minute on-period, and five minutes off-period.

Another way of understanding and quantifying the difference between neuroprotective therapy at 500 and symptom modulation at 522 is power density. The neuroprotective therapy may be generated between distant electrodes, 1, 2, or 3 cm, or more apart on a given lead, or using a first one or more electrodes as anodes on a first lead, and a second one or more electrodes as cathodes on a second lead, again with 1, 2 or 3 cm distance. The symptom modulation therapy at 522 may be more targeted to a smaller volume, using, for example, a monopolar output (one or more active electrodes at the target tissue, with a distant return electrode), or using bipolar configurations with the poles closely spaced, less than 2 cm, or less than 1 cm apart, for example. A numerical understanding of the difference may be to state that the peak power density for the symptom modulation 522 is at least twice that of the neuroprotective therapy, referenced to a volume of less than, for example, 1 cubic millimeter, or 0.2 cubic millimeters.

In another example, referring to FIGS. 8 and 9 together, the neuroprotective modulation is directed to a first target tissue 410, from a first lead 400. The symptom modulation signal may be directed to a second target tissue 424, from a second lead 422. In such an example, the first target tissue 410 may be selected for the neuroprotective modulation because it corresponds to a source of the malignant epileptic signaling. The second target tissue 424, one the other hand, may be selected for the symptom modulation due to it being a part of the networking of signals in the brain.

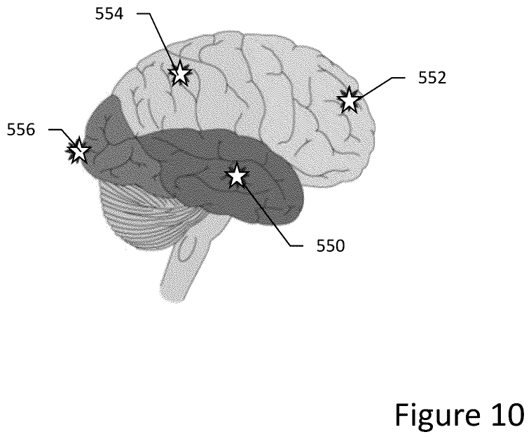

FIG. 10 graphically illustrates several targets on the superficial brain. The frontal lobe 552, parietal lobe 554, occipital lobe 556, and temporal lobe 558 may each be targets for therapy, either for neuroprotective therapy or for symptom modulation therapy. In some examples, the lobes as shown can be accessed by use of a sub-cranial electrode placed on the outside of the brain, similar to the lead/electrode 400 shown in FIG. 8. Paddle, linear, or even button electrodes and leads can be used, as desired. The electrodes positioned on the outside of such lobes may, in some examples, be used for neuroprotective therapy including each of optical and electrical outputs, and may (optionally) also be used as the sensing locations to identify EEG signals related to potential seizure onset, seizure onset, or seizure occurrence. In some examples, a second lead and/or electrode or electrode array may be positioned as well on the outside of the brain, where a first lead/electrode or electrode array is used for one purpose (neuroprotective and/or sensing) and the second lead/electrode or electrode array is used for a second purposes (sensing and/or symptom modulation). In other examples, as shown in FIG. 8, a second lead may be a DBS lead positioned inside the brain and used for interrupting malignant signals, for example.

In addition to the structures identified in FIG. 10, the system may also or instead target the hippocampus, the anterior thalamic nucleus, and/or the centro-median thalamic nucleus, as well as the cerebellum. These and other structures may be accessed using a deep brain stimulation lead. Target structures may also include seizure focus locations, if foci have been ascertained. Structures closer to the anterior midbrain and/or thalamus may be more amenable to optical and/or electrical signals generated by a device implanted in or a lead extending to a nasal sinus. Targets may include the internal medullary lamina, the dorsomedial nucleus, the intralaminar nuclei, the anterior nuclei, the centrum medium, the pulvinar or a portion thereof, etc. To the extent such structures are located at or near a lead, an operative mapping may be obtained using, for example, imaging of lead location and a brain atlas, allowing an understanding of which electrodes on a given lead or electrode structure are near/adjacent what anatomy. A user interface may then be used to allow a user to select electrodes according to the structure to which each is adjacent, if desired.

Programs may be defined, as is known in the neuromodulation arts, and called according to a schedule. This is one way to allow, for example, a daily delivery of neuroprotective modulation to the patient. Separate therapy control programs may be used for each of electrical and/or optical therapy outputs, or the two may be integrated into one therapy program. Integration may be useful to help synchronize outputs.

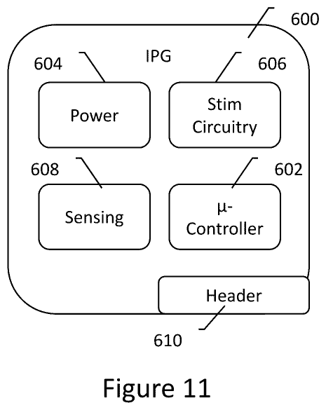

FIG. 11 shows additional details of a pulse generator. The IPG 600 may include separate circuits, sometimes referred to as operational circuitry, including a microcontroller 602 (which may also be implemented as part of a microprocessor if desired), which controls operations of the IPG at a high level. The IPG can include a power source 604, typically a battery (rechargeable or primary cell, as desired), though some systems may be adapted to operate without a battery by receiving power inductively or through other link (such as radiofrequency) and issuing therapy using the received power without long-term storage. Though not shown, if a rechargeable battery is used, or an externally powered system is implemented, a charging circuitry, such as those using inductive coils as are known in the art, would also be present.

A block of stimulation circuitry 606 is also provided. At a high level the stimulation circuitry 34 may include a plurality of current sources and current sinks (for a current-controlled system; a plurality of voltage sources may be used in voltage-controlled systems instead), and control circuitry including for example one or more analog ASICs, as well as switch arrays that implement steering instructions and/or electrode selections. U.S. Pat. No. 10,716,932 illustrates details for both current and planned future implementations of the stimulation circuitry 606, and is incorporated herein by reference. The IPG 600 will also house several blocks of memory, which may take any suitable form including, for example and without limitation, RAM, ROM and/or Flash memory. Such memory will store, for example, therapy programs to be used as well as operational functions for the system including for telemetry, self-diagnostics, charging control, battery management, etc.

A sensing block is also shown at 608, and may include any suitable combination of amplifiers, analog and/or digital filtering circuitry, analog-to-digital conversion circuitry, and other sensing-related circuits, such as comparators for determining when a sensed signal crosses a set threshold. Any suitable transducers may be included as well, such as for converting sensed/detected optical signals to electrical signals. An application-specific integrated circuit (ASIC) may be used as part of the sensing circuitry. A microcontroller may also be used and/or the sensing circuit 608 may be part of the microcontroller 602 if desired. One example uses analog filtering circuitry to remove DC components (below 1 Hz, for example) from incoming signals and a low pass filter to remove high frequency (above 1 kHz, for example) content in the analog domain; a bandstop filter may be used to remove 50 Hz/60 Hz line noise if desired as well. The remaining signal can be amplified and undergoes analog-to-digital conversion with sampling at a frequency set above the highest frequency of interest in the second signals (for example, sampling at 512 Hz or 1 kHz). Once in the digital domain further filtering may take place as well as various steps to maintain a reliable baseline or quiescent point. The final signal may be compared to set or varying thresholds for event detection, or may be compared to stored templates to analyze detected events, if desired. Frequency domain analysis may be performed as well, such as by using a fast Fourier transformation, principal components analysis, or wavelet transform. The signal, detected events, and/or frequency spectrum may then be analyzed to aid in identifying signal changes between ictal and inter-ictal periods, for example.

The IPG 600 may include a conductive outer housing that can serve as a return electrode or indifferent electrode during therapy delivery, as desired. A header 610 provides feedthrough circuitry allowing the IPG 600 to couple to one or more leads, with separate electrical connections to each of the electrodes of such leads. Optical sources may be part of the stimulation circuitry 606 as well or, in the alternative, drivers may be provided for generating power signals for optical transducers provided in the leads or on/integrated with paddle electrodes or directional electrodes as described above. The header 610 will include electrical and/or optical connections as needed. The header 610 and housing provide a hermetic sealed environment for the operational circuitry 602, 604, 606 and associated memory (not shown), all of which may be coupled by various interconnections (wires and/or buses) that are omitted from the drawing. Telemetry circuitry, such as using Bluetooth and/or Medradio wireless technology, or other wireless communication modes, may be used as well as with associated antennae and other circuitry.

In some examples in the prior art, a plurality of programs can be set for therapy delivery by the IPG 600. Each program may operate according to a schedule and individual program parameters. A program scheduler may determine whether and when stored therapy programs are called for execution, and may be encoded in stored instructions executed by the micro-controller 602 and/or stimulation circuitry 606.

Each of these non-limiting examples can stand on its own, or can be combined in various permutations or combinations with one or more of the other examples.

The above detailed description includes references to the accompanying drawings, which form a part of the detailed description. The drawings show, by way of illustration, specific embodiments. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls. In this document, the terms “a” or “an” are used, as is common in patent documents, to include one or more than one, independent of any other instances or usages of “at least one” or “one or more.” Moreover, in the claims, the terms “first,” “second,” and “third,” etc. are used merely as labels, and are not intended to impose numerical requirements on their objects.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Further, in an example, the code can be tangibly stored on one or more volatile, non-transitory, or non-volatile tangible computer-readable media, such as during execution or at other times. Examples of these tangible computer-readable media can include, but are not limited to, hard disks, removable magnetic or optical disks, magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. § 1.72 (b), to allow the reader to quickly ascertain the nature of the technical disclosure. It is submitted with the understanding that it will not be used to interpret or limit the scope or meaning of the claims.

Also, in the above Detailed Description, various features may be grouped together to streamline the disclosure. This should not be interpreted as intending that an unclaimed disclosed feature is essential to any claim. Rather, innovative subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the protection should be determined with reference to the appended claims, along with the full scope of equivalents to which such claims are entitled.

What is claimed is:

1. A method of treating a patient comprising:
a) optically and electrically modulating a first neural target of the patient to provide neural protection to reduce progression of epilepsy;
b) identifying a triggering event indicating an elevated likelihood of actual or impending epilepsy seizure; and
c) in response to the identifying, electrically modulating a second neural target of the patient to mitigate effects of the epilepsy seizure.

2. The method of claim 1, wherein step a) is performed using a cortical electrode positioned superficial to the brain; and step c) is performed using a deep brain stimulation electrode positioned inside the brain.

3. The method of claim 1, wherein step a) is performed on a periodic basis, and steps b) and c) are performed only when a triggering event is identified.

4. The method of claim 1, wherein electrical modulating in step a) is performed using two electrodes that are more than at least 2 cm apart, and step c) is performed using two electrodes that are less than one cm apart.

5. The method of claim 1, wherein step a) is performed at a lower power density than step c).

6. The method of claim 1, wherein step a) is performed with a lower output amplitude, duty cycle, pulse width, or frequency than step c).

7. The method of claim 1, wherein step b) is performed by sensing electrical signals from the brain of the patient, analyzing the sensed electrical signals, and finding a divergence from a brain signal captured during an inter-ictal time period.

8. The method of claim 1, wherein step c) is performed by:
initiating electrical modulation of the second neural target;
analyzing the trigger condition and continuing the electrical modulation of the second neural target until the trigger condition ceases.

9. The method of claim 1, wherein step c) is performed by:
initiating electrical modulation of the second neural target;
continuing the electrical modulation of the second neural target until a stop-therapy condition arises.

10. The method of claim 9, wherein the stop-therapy condition includes a patient indication that a seizure is not occurring.

11. The method of claim 9, wherein the stop-therapy condition includes a timeout.

12. The method of claim 9, wherein the stop-therapy condition includes a determination that the patient no longer shows an elevated likelihood of actual or impending epilepsy seizure.

13. The method of claim 1, wherein step c) includes interrupting step a) and preventing step a) until step c) is completed.

14. The method of claim 1, wherein the first target is associated with generation of malignant electrical signals that can lead to epilepsy seizure, and the second target is associated with transmission of the malignant electrical signals.

15. The method of claim 1, wherein the first target is a seizure focus, and the second target is a part of the hippocampus.

16. The method of claim 1, wherein the first target is on or in the cerebral cortex, and the second target is a part of the hippocampus.

17. A system for treating a patient having a seizure condition comprising:
a cortical lead adapted for positioning inside the skull of a patient and on the outside of the brain, the cortical lead including each of an electrode and an optical output device;
a deep brain stimulation lead adapted for positioning inside the brain of a patient near the hippocampus or near a seizure focal location;
a pulse generator coupled to each of the cortical lead and the deep brain stimulation lead, the pulse generator comprising a control circuitry, a sensing circuitry, and a pulse generator circuit, the control circuitry configured to:
a) use the pulse generator circuit and the cortical lead to issue each of first optical outputs and first electrical outputs to modulate a first neural target of the patient to provide neural protection and reduce progression of epilepsy;
b) use the sensing circuitry for identifying a triggering event indicating an elevated likelihood of actual or impending epilepsy seizure; and
c) in response to the identifying, use the pulse generator circuit and the deep brain stimulation lead to issue second electrical outputs to modulate a second neural target of the patient to mitigate effects of the epilepsy seizure.

18. The system of claim 17, wherein the control circuitry is also configured, in step c), to use the pulse generator circuit and the deep brain stimulation lead to issue second optical outputs to modulate the second neural target of the patient.

19. The system of claim 17, wherein the first neural target includes a portion of the cerebral cortex.

20. The system of claim 17, wherein the second neural target is the hippocampus.
